(19) United States
(12) Patent Application Publication
(10) Pub. No.: US 2009/0004275 A1
(43) Pub. Date: Jan. 1, 2009

(54) Composition for Delivery of an Active Agent

(75) Inventors: Glen Patrick Martyn, Nottingham (GB); David St. John Coghlan, Nottingham (GB)

Correspondence Address: STERNE, KESSLER, GOLDSTEIN & FOX P.L.L.C., 1100 NEW YORK AVENUE, N.W., WASHINGTON, DC, 20005, US

(73) Assignee: Quadrant Drug Delivery Limited, Ruddington (GB)

(21) Appl. No.: 11/658,653

(22) PCT Filed: Jul. 29, 2005
(86) PCT No.: PCT/GB05/02977

§ 371 Date: Jan. 18, 2008

(30) Foreign Application Priority Data
Jul. 29, 2004 (GB) 0416861.3

(51) Int. Cl. A61K 9/00; A61K 38/02; A61K 38/28

(52) U.S. Cl. 424/488; 514/2; 514/3

(57) ABSTRACT

A composition for oromucosal delivery of a biologically active agent, which composition releases the active agent within 5 minutes when applied to an oramucosal surface in use, comprises a biologically active agent and a matrix-former. The biologically active agent may be present as an ion pair complex.

This invention relates to a composition for oromucosal delivery of a biologically active agent.

As scientists search for new drugs to treat serious human diseases, peptides, proteins, and other (larger) macromolecules are becoming increasingly more important. Often, these macromolecules have to be injected due to poor bioavailability by other routes. However, patient compliance is often low for such injectable drugs and their development is expensive. For decades, researchers in the field of drug delivery have sought ways in which to dose larger molecules like peptides and proteins orally. However, the low pH of the stomach and peptidases and proteolytic enzymes present throughout the gastrointestinal tract are responsible for the rapid and extensive degradation of these drugs.

Alternative routes such as transmucosal delivery via the lung, nose, and buccal tissue are being developed. The buccal mucosa offers a near ideal portal through which macromolecules might enter the body. There is a great potential for the rapid absorption of drugs into the circulatory system due to the large surface area of the buccal mucosa and the direct access to a rich network of blood vessels. This route also avoids the hepatic first pass metabolism with a related increase in bioavailability, as well as side effects related to gastrointestinal absorption.

A wide variety of compounds have been reported in the literature as oral mucosal penetration enhancers, most notably bile salts, surfactants and chelators. Yang et al, Chem Pharm Bull (Tokyo), 2002 June; 50(6): 749-53, “Phospholipid deformable vesicles for buccal delivery of insulin” describes phospholipid deformable vesicles for the buccal delivery of insulin. These vesicles, comprising a combination of phosphatidylcholine, cholesterol and sodium deoxycholate, were administered to rabbits and demonstrated relative bioavailability of between 16% and 20%, compared with subcutaneous administration of insulin solution.

Another study, [http://www.ijpe.org/Jul2002/Article11Page02.html] reported similar relative bioavailabilities when investigating the effects of various transmucosal absorption enhancers on insulin permeation through hamster and rabbit buccal membranes in vitro and in vivo. The concomitant administration of sodium deoxycholate, sodium lauryl sulfate, lecithin and Brij 78 was the most effective combination of absorption enhancers, rendering buccal insulin one-fifth to one-fourth as effective as sub-cutaneous insulin.

U.S. Pat. No. 6,613,358 describes sustained release compositions for pharmaceutical use. The compositions are based on a biocompatible polymer and a pharmaceutical substance in a hydrophobic ion complex with an amphiphilic material. A similar disclosure of compositions comprising a hydrophobic ion pair complex (“HIP”) can be found in U.S. Pat. No. 5,981,474. There is no practical teaching in the above documents of buccal or oromucosal delivery of an active substance.

EP-A-414080 describes compositions for the delivery of insulin that comprise an alkali metal cocoate. The compositions may be formulated for buccal delivery as films containing hydroxypropyl cellulose that are cast and dried at room temperature. The cast film can dissolve relatively slowly and the cocoate adversely affects the taste of the composition.

WO 02/066016 describes a mucoadhesive pharmaceutical preparation in the form of a polymer matrix containing an active material. The document discloses the delivery of small molecules and does not address the problems associated with the delivery of proteins such as insulin.

U.S. Pat. No. 6,432,383 discloses a mixed micellar pharmaceutical formulation comprising insulin for use in a metered dose inhaler. The inhaler is for buccal delivery of insulin, but in practice many puffs of the inhaler are required to give a suitable dose. This creates problems with patient compliance.

WO 2004/075877 discloses a transmucosal delivery system. There is no mention in the document of adjusting the pH to form ion pairs.

There remains a need to improve on the above formulations and methods in order to achieve a pharmacokinetic profile similar to that obtained via parenteral administration, particularly with reference to speed of onset. Also, there is a need to achieve an easier format and/or a system that allows better patient compliance and/or improved taste.

Accordingly, the present invention provides a composition for oromucosal delivery of a biologically active agent, which composition releases the agent within 5 minutes when applied to an oromucosal surface in use, wherein the composition comprises a biologically active agent and a matrix-former, and the biologically active agent is present as an ion pair complex. Preferably, the composition dissolves or disperses within 5 minutes. It will be appreciated that the agent is incorporated in the composition.

In another aspect, the invention provides a composition for oromucosal delivery of a biologically active agent, which composition releases the agent within 5 minutes when applied to an oromucosal surface in use, wherein the composition comprises a biologically active agent and a matrix-former, and the composition is formed by freeze drying a solution, dispersion or emulsion comprising the biologically active agent and the matrix-former. Preferably, the composition dissolves or disperses within 5 minutes.

The invention also contemplates, in another embodiment, a composition for oromucosal delivery of a biologically active agent, which composition dissolves or disperses within 5 minutes when applied to an oromucosal surface in use, wherein the composition comprises a biologically active agent and a matrix-former.

Another aspect of the invention is a composition of the invention for use in medicine.

A further aspect of the invention is a method of treating a condition or disorder which comprises administering an effective amount of a composition of the invention to a patient in need thereof.

Yet another aspect of the invention is the use of the composition of the invention in the manufacture of a medicament for the treatment of a condition or disorder, such as diabetes.

In particular, the invention relates to drug delivery systems, especially suitable for oromucosal delivery. The compositions of the invention may be formed as relatively large film strips or sheets and subsequently cut into uniform dosage units, or lyophilised as individual dosage units, each dosage unit being substantially uniform in content and having distributed therein a matrix-former and a biologically active agent, optionally present as an ion-pair complex, more preferably of the type referred to in the art as a hydrophobic ion-pair complex.

The present invention provides improved dosage forms for the non-invasive delivery of biologically active molecules. The medicinal preparations according to the invention typically release the incorporated active agent (and preferably disintegrate or dissolve) after having been inserted into an aqueous medium (e.g., water) or in body fluids, preferably the oromucosal surface in use, i.e. within up to 5 minutes after insertion, preferably less than 4 minutes, more preferably less than 3 minutes, such as less than 2 minutes, for example less than 1 minute. The method of release is preferably via dissolution or disintegration of the dosage form but may include other forms of release, such as hydration and subsequent release, e.g., chewing gum.

Oromucosal administration of the compositions may involve administering an effective dose of the biologically active agent as a single dose (i.e., the composition may be in unit dosage form), or the effective dose may be administered in a plurality of smaller doses over a period of time, the smaller doses being sufficient to be equivalent to that of a single dose.

The composition of the invention may be produced by a number of different routes. In one route, the biologically active agent is dissolved or dispersed in an aqueous medium further comprising the matrix-former and optionally other excipients or processing agents. This solution, dispersion or emulsion is then cast as a film and dried. The film may be dried as unit dosages or as a sheet or roll from which individual dosages are removed by the patient or at the time of manufacture.

In another route, the biologically active agent may be processed with a further ionic agent to form a hydrophobic ion pair (HIP) complex. This complex may then be dissolved, dispersed or emulsified in a solvent system comprising the matrix- or film former(s), optionally with further excipients and processing agents, and then cast as a film and dried. The film may be dried as unit dosages or as a sheet or roll from which individual dosages are removed by the patient or at the time of manufacture.

In another route, the HIP complex described above may then be dissolved, dispersed or emulsified in a solvent system comprising the matrix-former (also referred to herein as the matrix-forming agent(s)), optionally with further excipients and processing agents, and then lyophilised as unit dosage forms as described in WO 00/50013.

As an alternative or in addition to the HIP complex, the biologically active compound may be associated or otherwise complexed with a surfactant or detergent, for example in the form of micelles or liposomes. Preferred surfactant or detergent systems comprise an alkali metal lauryl sulfate, a pharmaceutically acceptable edetate, at least one alkali metal salicylate, and at least one micelle forming compound selected from the group consisting of lecithin, hyaluronic acid, pharmaceutically acceptable salts of hyaluronic acid, octylphenoxypolyethoxyethanol, glycolic acid, lactic acid, chamomile extract, cucumber extract, oleic acid, linolenic acid, borage oil, evening primrose oil, trihydroxy oxo cholanylglycine, glycerin, polyglycerin, lysine, polylysine, triolein and mixtures thereof. Preferably, each of said sulfate, edetate and salicylate is present in a concentration of from 0.1 to 10 w/w % (more preferably 1 to 10 w/w %) of the total formulation. More preferably, each micelle forming compound is present in a concentration of from 1 to 10 w/w % of the total formulation, and the total concentration of sulfate, edetate, salicylate and micelle forming compounds is less than 50 w/w % of the formulation. Preferably, the alkali metal lauryl sulfate is sodium lauryl sulfate. A preferred edetate is an alkali metal edetate. The alkali metal salicylate is preferably sodium salicylate. The micelle forming compound is preferably lecithin, lecithin in combination with hyaluronic acid, evening of primrose oil or borage oil. A particularly preferred formulation comprises a combination selected from the group consisting of: i) sodium lauryl sulphate, sodium salicylate, disodium edetate, saturated phospholipid, and sodium hyaluronate; ii) sodium lauryl sulphate, sodium salicylate, disodium edetate, lecithin, and sodium hyaluronate; iii) sodium lauryl sulphate, sodium salicylate, disodium edetate, sodium hyaluronate, and evening of primrose oil; iv) sodium lauryl sulphate, sodium salicylate, disodium edetate, saturated phospholipid, and bacitracin, v) sodium lauryl sulphate, sodium salicylate, disodium edetate, saturated phospholipid, sodium hyaluronate and bacitracin; and vi) sodium lauryl sulphate, sodium salicylate, disodium edetate, sodium hyaluronate, oleic acid and gamma linoleic acid. The micelle formulation preferably further comprises water.

The term “oromucosal” refers to a dosage form that is to be placed under the tongue, against the cheek or anywhere else in the oral cavity that allows the active ingredient to come in contact with the mucosa of the oral cavity or the pharynx of the patient, in particular the sublingual and buccal regions.

Compositions of the invention are preferably adapted for oromucosal delivery.

In a preferred embodiment, the composition may be of any desired size and shape as is known in the art to deliver a preferred dosage of the bioactive agent and to fit within the buccal cavity either on the mucosal surface of the cheek, between the cheek and gum surfaces or under the tongue. Suitable product forms for the compositions include, oral patch, gum, filn, strip, paper, suppository, or pessary dosage form or even powder or granules. Particularly preferred dosage forms include films and wafers. One or a number of films or wafers may be used for each dosage administration, if desired. The thickness of such wafers is typically 5 μm to 10 mm, preferably 30 μm to 2 mm, and with particular preference 0.1 mm to 1 mm. The wafers may advantageously be of round, oval, elliptic, triangular, quadrangular or polygonal shape, but may also be of any rounded shape. Film thickness should preferably be about 5 microns to about 200 microns (e.g., 10 microns to 200 microns) and especially preferred thicknesses are about 20 microns to about 75 microns. A circular or square film of about 1-20 cm² surface area can be particularly useful in delivering the biologically active agent. It may also be appreciated that the resulting film may be prepared with a removable backing to protect the film during storage and handling.

The compositions of the invention may also include various excipients or processing aids, including, without limitation, buffers, lubricants, disintegrators, plasticizers, binders, absorbents, diluents, taste-masking agents, surfactants, emulsifying agents, thickening agents, cooling agents, saliva-stimulating agents, sweetening agents, antimicrobial agents and combinations thereof, as needed or desired, in accordance with established pharmaceutical industry practice.

Suitable buffering agents include phosphates, carbonates, tartrates, borates, citrates, acetates, maleates and amino acids such as glycine, histidine. Combinations of such buffering agents are particularly desired.

Plasticizers are used to preserve film flexibility which is a desired property for sublingual applications. Suitable plasticisers include, but are not limited to: polyols, glycerin, erythritol, sorbitol, mannitol, propylene glycol, polyglycerols, short chain aliphatic monoglycerol esters, and polyethylene oxides.

According to a preferred embodiment, the compositions according to the invention contain at least one flavouring substance and/or at least one sweetener and/or at least one plasticizer. The uptake of active substance can furthermore be improved by means of substances stimulating the blood flow which can be added to the preparations according to the invention. Among these are, in particular: menthol, eucalyptol, ginkgo extract, geranium oil, camphor, spearmint oil, oil of juniper and rosemary. These blood flow-stimulating substances may be used singly or in combination.

The invention also includes preparations which are present in the form of thin, solid foams. Wafers in the form of thin foams are advantageous since they quickly adhere to the mucosa due to their large specific surface, and since they on the other hand also disintegrate quickly. The density of these solidified foams is preferably between 0.01 g/cm³ and 0.10 g/cm³, with particular preference between 0.08 g/cm³ and 0.4 g/cm³, and with greatest preference between 0.1 g/cm³ and 0.3 g/cm³. The above-mentioned foams may be produced by introducing and dispersing gases with the aid of special foam beating devices, or by dissolving gas under pressure and subsequent relaxation of the solution and most preferably by microwave-vacuum drying.

The compositions preferably dissolve or disperse in the oral cavity (or in water) in less than 5 minutes, preferably less than 4 minutes, preferably less than 3 minutes, most preferably less than 2 minutes, and even more preferably less than 1 minute.

The compositions of the invention may be multi-layered (i.e., comprising two or more layers), for example multi-layered unit dose forms. These product forms are particularly preferred when there is an advantage to be gained by having two or more layers of the same or different film matrices to constitute the dose form. For example, it may be desirable to have one layer consist of a fast dissolving substance which contains the drug of interest to be placed against the target tissue with a second outer layer composed of a material with slow or little dissolution by the saliva and which acts as a protective coating for the inner layer. Another embodiment comprises one layer which releases the drug of interest rapidly and another layer which releases it slowly so as to provide the patient with immediate and sustained drug delivery into the target oral region. The ability to easily construct a layered dose form allows for a great deal of flexibility in drug delivery. Preferred polymers for the slow release layer are poly(lactic acid) homopolymers, including poly(l-lactic acid) and poly(d-lactic acid), poly(glycolic acid) homopolymer, polyanhydrides, such as poly(sebacic acid), poly(carboxyphenoxyhexane), polybutyrates and cellulosic polymers such as polyhydroxypropyl ethylcellulose. In a further embodiment, said slow release component may be formulated as a particulate and then dispersed in the solvent comprising the rapid release component(s) followed by subsequent removal of the solvent. This slow release component preferably demonstrates mucoadhesion.

The matrix-former(s) constitute(s) at least 0.1% by weight and maximally 99% by weight (e.g., 0.3% to 99% by weight), more preferably 1 to 80% by weight (e.g., 5% to 80% by weight), most preferably 10 to 50% by weight, each value being relative to the weight of the entire composition. The mucoadhesive properties as well as the disintegration properties are determined substantially by the type of the matrix-former (e.g., matrix-forming polymer(s)), as well as by the relative proportions and combinations of these polymers in the preparation.

Substances intended for use, singly or in combination, as the matrix-former include, but are not limited to: water-soluble or dispersible hydrocolloidal gums such as gum arabic, xanthan gum, acacia, locust bean gum, pectin, alginates, guar gum, carrageenan, curdlan, beta-glucans, dextrans and gum tara; hydrolytically or chemically modified oligosaccharides and polysaccharides such as chitosan, low dextrose equivalent (DE) starches, maltodextrins, dextrins, dextrans, polydextrose, high amylose starch, lipophilic substituted starches (such as hydroxyethyl starch), pregelatinized starches, and propylene glycol alginate; water-soluble cellulose ethers such as hydroxypropyl cellulose, sodium carboxymethylcellulose, hydroxypropyl methylcellulose, methylcellulose and ethylcellulose; water-soluble synthetic polymers such as polyvinyl pyrrolidone, polyethylene glycols, and polylactic acid; soluble or dispersible proteins such as gelatin, prolamines (zein, gluten), whey protein isolates, casein and salts thereof, soy protein isolates, and albumins; and combinations thereof. Examples of other polymeric materials include polyvinyl alcohol, polyacrylic acid, polyvinyl pyrrolidone, poly(meth)acrylate, poly(meth)copolymers and combinations thereof and low molecular weight carbohydrates such as maltose, maltitol, mannitol, sorbitol, erythritol, dextrose, sucrose, trehalose, palatinit, lactose and lactitol. The above are provided as exemplary ingredients and are not intended to limit the compositional scope of the film-forming matrix.

A preferred matrix-former is pullulan. Pullulan (CAS Reg. No. 9057-02-7) is an extracellular polysaccharide [an alpha-D-glucan] consisting predominantly of repeating maltotrioses linked by alpha-1,6-glucosidic bonds. It is excreted by the fungus Aureobasidium pullulans. Molecular weights for pullulan range from 8,000 to 2,000,000 daltons depending on the growth conditions of the organism. Pullulan is non-hygroscopic and non-reducing, is soluble in hot and cold water and is generally insoluble in organic solvents. It has a glass transition temperature of over 150° C. Pullulan readily forms films which are mucoadhesive, impermeable to oxygen, thermally stable, anti-static, and elastic. Due to the relatively low molecular weight, water solubility is very high and attaining useful viscosities for film forming requires a high content of pullulan. Therefore, in one aspect of the present invention there is included a film or wafer composition which comprises pullulan and a water-soluble polymer, wherein the weight ratio of pullulan to water soluble polymer is about 50:1 to about 0.1:10. Water-soluble polymers useful in the present invention include cellulosic materials, gums, proteins, starches, and combinations thereof, as cited above.

The term “hydrophobic ion-pairing (HIP)” refers to the interaction between amphiphilic materials (also referred to herein as lipophilic agents or species) e.g. detergents, which interact with the biologically active agent(s), such as proteins, other polypeptides and nucleic acids. “HIP complex derivatives” are substances modified by formation of a hydrophobic ion-pair. The amphiphilic material interacts with an oppositely charged compound, such as a polypeptide or nucleic acid.

Preferably, the amphiphilic material and the biological agent (also referred to herein as pharmaceutical substance, drug or pharmaceutical agent) have oppositely charged ionic portions which associate to form an ion pair complex—thus, the pharmaceutical substance may comprise an anionic portion which associates with a cationic portion of the amphiphilic material or a cationic portion which associates with an anionic portion of the amphiphilic material.

Preferably, the pH is adjusted to enhance ion-pair formation, for example by generating ionised (e.g., protonated, deprotonated or otherwise disassociated) forms of the compounds present.

For a basic drug, a lipophilic species according to the invention is, for example, a fatty acid or another lipophilic species. For a basic drug, a lipophilic species according to the invention may, for example, be one or more of the following fatty acids, or long-chain alkyl sulfonic acids, or a long-chain alkyl sulfuric acids: caproic, caprylic, cupric, lauric, myristic, palmitic, stearic, arachidic, behenic, lignoceric, myristoleic, palmitoleic, oleic, gadoleic, erucic, ricinoleic, linoleic, linolenic, licanic or arachidonic.

Anionic detergents are preferred for use in the formation of HIP complexes.

As used in the present invention, the term “anionic detergents” encompasses any hydrophobic material that is a salt of an acid, such as fatty acids (e.g., straight chain carboxylic acids containing from 10 to 24 carbon atoms) including sulfates, sulfonates, phosphates, and carboxylates. Sulfates are the salts of the stronger acids in this series and, therefore, the most efficient at forming ion pairs.

Preferred examples of anionic amphiphilic materials include sulfates, sulfonates, phosphates (including phospholipids such as DPPG, DPPA, DLPG), carboxylates, and sulfosuccinates. Some specific anionic amphiphilic materials useful with the present invention include: sodium dodecyl sulfate (SDS), bis-(2-ethylhexyl) sodium sulfosuccinate (AOT), cholesterol sulfate and sodium laurate. Particularly preferred anionic amphiphilic materials are SDS and AOT.

For an acidic drug, a lipophilic species according to the invention is, for example, a fatty amine or another lipophilic species. For an acidic drug, a lipophilic species according to the invention is, for example, cetrimide, oleamidopropyl dimethylamine, didecyldimethyl ammonium chloride, quaternary surfactants, cetylpyridinium chloride, hexetidine, benzalkonium chloride, arginine and cholesterol esters, carbamates, carbonates and ketals and the following fatty amines and acid amides: caproic, caprylic, capric, lauric, myristic, palmitic, stearic, arachidic, behenic, lignoceric, myristoleic, palmitoleic, oleic, gadoleic, erucic, ricinoleic, linoleic, linolenic, licanic, arachidonic and/or clupanadonic.

The size of HIP complexes is controlled by controlling the rates of the mixing of a solution comprising the biologically active agent and the addition of an amphiphilic material, such as anionic or cationic detergent, to the solution. The particle size of the HIP complex which is formed in water will depend on the degree of agitation of the solution and the rate of counterion addition. The smallest particles are produced with high shear being applied to an aqueous solution and slow addition of amphiphilic material, e.g. by employing a high speed or high pressure homogenizer or sonication.

The HIP complex may also be solubilised by the addition of a further secondary surfactant. Preferred secondary surfactants include non-ionic surfactants such as polyoxyalkylene (e.g., polyoxyethylene) ethers having C₄ to C₂₄ alkyl chains, for example Brij 35.

Particularly useful as biologically active agents are macromolecules such as polymers, nucleic acids, proteins, polypeptides, natural peptides, synthetic peptides, peptide mimetics, hormones, D and L amino acid polymers, nucleotides, oligonucleotides and nucleic acids, including DNA and RNA, protein nucleic acid hybrids, genes, antisense RNAs, ribozymes, plasmids and small molecules and physiologically active analogs thereof. Particularly relevant actives include enzymes, blood factors, biopharmaceuticals, growth hormones, growth factors, insulin, humanized or chimaeric monoclonal antibodies, interferons, interleukins and cytokines. Leuprolide and HGH (1-217) are examples of biologically active agents. Insulin is particularly preferred. Combinations of one or more biologically active agent may be used.

Other suitable active agents include immunogens such as vaccines. Suitable vaccines include, but are not limited to, live and attenuated viruses, nucleotide vectors encoding antigens, bacteria, antigens, antigens plus adjuvants, haptens coupled to carriers. Particularly preferred are vaccines effective against Alzheimer's disease, plague, anthrax, botulinum, diphtheria, tetanus, pertussis, botulinum, cholera, Dengue, Hepatitis A, B, C and E, hemophilus influenza b, herpes virus, Hylobacterium pylori, influenza, Japanese encephalitis, meningococci A, B and C, measles, mumps, papilloma virus [HPV], pneumococci, polio, rubella, rotavirus, respiratory syncytial virus, Shigella, tuberculosis, yellow fever and combinations thereof. Vaccines may also be produced by molecular biology techniques to produce recombinant peptides or fusion proteins containing one or more portions of a protein derived from a pathogen. Also suitable are allergens.

The term “allergen” refers to any naturally occurring protein or mixtures of proteins that have been reported to induce allergic, i.e. IgE mediated reactions upon their repeated exposure to an individual. Examples of naturally occurring allergens include pollen allergens (tree, weed, herb and grass pollen allergens), mite allergens (from e.g. house dust mites and storage mites), insect allergens, animal allergens from e.g. hair and dander from e.g. dog, cat, horse, rat, mouse, etc., fungi allergens and food allergens. The allergen may be used in the form of a purified allergen, an allergen extract, a modified allergen or a recombinant allergen or a recombinant mutant allergen. Normally an allergen extract contains at least 10% protein of the dry matter content—the remainder consists of components such as lipids, carbohydrates, or bound water which originates from the biological allergen source.

Suitable ionizable pharmaceutical agents for use in the invention may include one or more of the following: dihydroergotamine, fentanyl, sufentanil, lidocaine, alfentanil, lofentanil, carfentanil, pentobarbital, buspirone, ergotamine, bisphosphonates, alendronic acid, nalbuphine, prostaglandins, bupropion, metformin, diethylcarbamazine, kamadol, heparin or a heparin derivative, amoxicillin, gabapentin, econazole, aspirin, prostaglandin, methylsergide, ergonovine, endorphins, enkephalins, oxytocin, opiates, fibrates, barbiturates, albuterol, atropine, scopolamine, selegiline, lamotrigine, pilocarpine, timolol, nicotine, cocaine, novocaine, amphetamines, cannabinoids, caffeine, heparin and its derivatives, clorazepic acid, methylphenidate, chlorpromazine, ketamine, epinephrine, estropipate, naloxone, nalkexone, levothyroxine, folic acid, nicotinic acid, pantothenic acid, retinoic acid, risperidone, furosemide, labetalol, metoprolol, nadolol, isoproterenol, terbutaline, bupivacaine, prilocalne, loratadine, chloropheniramine, clonidine, or tetracaine.

In one preferred embodiment, the pharmaceutical agent is nicotine.

In a further preferred embodiment, the pharmaceutical agent is a 5-HT agonists for use in the treatment of migraine, e.g. menstrually-associated migraine. Suitable examples include, without limitation, sumatriptan, naratriptan, rizatriptan, eletriptan, almotriptan, zolmitriptan, frovatriptan, F 11356, pharmaceutically acceptable salts thereof, and combinations thereof.

A particularly preferred agent for use in this invention is insulin. The insulin may be human recombinant or of animal origin for delivery to animals or other mammals. Insulin analogs such as insulin aspart, insulin lispro, insulin glargine, insulin NPH, insulin ultralente may also be used. The newer soluble analogs such as glargine and aspart may be particularly suitable for such oromucosal delivery. The amount of insulin in the compositions of this invention is typically a quantity that provides an effective reduction of glucose. In consideration of the fact that the bioavailability of any active substance can never be 100%, that is to say the administered dose of the active drug is not completely absorbed, it is preferable to incorporate a slightly larger amount than the desired dosage. For example, the insulin dose in the current invention may be in the region of 10 IU/kg.

Compositions of the invention may be for use as pre-prandial insulin. Compositions of the invention may be used in the treatment and/or management of Type I and/or Type II diabetes.

One aspect of the invention is to present insulin in a form favourable for its transport across the buccal mucosa. Optimisation of the pH of the formulation is critical for maximising the absorption of the insulin. The extent of hypoglycaemia induced by the buccal administration of insulin as a function of the pH exhibits a profile, which is comparable to the pH-solubility profile of insulin. Insulin contains six basic groups and four acidic groups. By lowering the pH to 2.5, all of the acidic groups become protonated producing an overall charge of +6. The composition can comprise an ion-pair forming reagent wherein the mole ratio of ion-pair forming reagent to insulin is from about 0.1:1 to about 1:10 The ion-pair-forming reagent is added to increase the lipophilicity of the insulin and thereby increase its membrane permeability. Increasing the drug's lipophilicity may also provide some protection of the drug from enzymatic deactivation. It is important to keep the counter-ion effect minimal by adding a “buffer” to keep pH below the isoelectric point, ideally less than pH 4. Such a low pH will maintain the ion-pair complex. Common oral acidulants such as citric acid, tartaric acid, etc. are suitable. Another excipient, which may be added for both its effect on maintaining acidity and prevention of collapse of the freeze-dried plug, is glycine. Combinations of such buffering agents and collapse inhibitors are particularly preferred.

Preferred amphiphilic agents for use in forming HIP complexes with insulin are AOT, SDS and DPPG. Preferred weight ratios of amphiphilic agent to biologically active agent are in the range of from 2:1 to 500:1, more preferably 5:1 to 300:1, even more preferably 10:1 to 200:1, such as 10:1 to 100:1.

The HIP complex may be loaded alone or in conjunction with bile salts, preferably sodium deoxycholate (SDC) and lecithin (L), or other enhancers to further increase absorption, via potential liposome andl or micelle formation. For human consumption, aspartame may be added [to help with peptidase activity as it is a dipeptide] as sweetener. Other potential absorption promoters include sugars such as dextran sulphate and the cyclodextrins, particularly dimethyl- and sulfobutyl-cyclodextrin. Cyclodextrins may complex with ion-paired or free insulin, enhance absorption, and also stabilise protein via high glass transition temperature (Tg). Aminopeptidase inhibitors in buccal bioadhesive delivery systems may be desirable to improve buccal bioavailability. Such inhibitors included EDTA and sodium deoxycholate.

In yet another embodiment, the HIP complex is dissolved in a suitable organic solvent system comprising an amphiphilic polymer, such as a low molecular weight hydroxypropyl cellulose or hydroxypropyl methylcellulose, and dried to produce a film, wafer or similar dosage form of the invention, preferably lyophilized from tert-butanol or air or microwave-vacuum dried. The advantage of this system is that the HIP complex is presented as a solid solution thereby improving membrane permeability over the dried HIP dispersion or emulsion formats.

Microwave drying is extremely efficient for water removal but requires considerable control be exercised to prevent thin film distortion. Typically, microwave drying for this application is used in combination with conductive and/or forced air drying. Such microwave drying is useful because drying initiates in the middle portions of the film. Programming from high energy to low energy operating levels may be employed as the drying progresses to avoid or minimize overheating and distortion of the films. Another drying technique for obtaining the films of the present invention is controlled radiation drying, in the absence of uncontrolled air currents, such as infrared. Desirably, the drying of the film will occur within about ten minutes or fewer, or more desirably within about five minutes or fewer. The drying includes applying heat to the bottom of the carrier surface.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

The following non-limiting examples illustrate the invention and do not limit its scope in any way. In the examples and throughout this specification, all percentages, parts and ratios are by weight unless indicated otherwise.

### EXAMPLES

### Example 1

To a rapidly agitated 50 ml solution comprising insulin (2 mg/ml) in 10 mM sodium acetate buffer, pH 2.5, is slowly added by burette 3.5 ml [4.5 molar equivalents] of AOT (10 mg/ml) in water, resulting in efficient precipitation of a HIP complex of less than 10 microns. To this aqueous dispersion is slowly added 5 ml of a 5 mg/ml sodium deoxycholate solution and then 10 ml of a 10 mg/ml pullulan solution. This is gently stirred until dissolved or fully hydrated. To half this dispersion is added a further 500 mg mannitol, allowed to dissolve and the complete feedstock is then added to 1×1 cm blister wells in 1 ml aliquots. The blisters are loaded into a Heto freeze-drier and frozen on the shelf held at −32° C. before turning on the vacuum and lyophilising the frozen solution for 24 h (ramping from −20° C. to 30° C.) to yield non-friable intact porous wafers in the blister packs which dissolve instantaneously in water at room temperature (15-20° C.).

The other half of the primary feedstock is cast onto a heated metal plate at 40° C. and rapidly air-dried to produce a thin flexible film which is then cut into individual dosages of 1×1 cm.

### Example 2

DPPG was suspended at a concentration of 10 mg/ml in water at 40° C. and stirred to achieve a clear viscous consistency (about 30 minutes). To a rapidly agitated 50 ml solution comprising insulin (2 mg/ml) in 10 mM sodium acetate buffer, pH 2.5, is slowly added by burette 10.26 ml of this DPPG solution (8 molar equivalents), resulting in efficient precipitation of a HIP complex of less than 10 microns. To this aqueous dispersion is slowly added 5 ml of a 5 mg/ml sodium taurocholate solution and then 10 ml of a 10 mg/ml pullulan: HPC blend solution. This is gently stirred until dissolved or fully hydrated. To half this dispersion is added a further 500 mg hydroxypropyl beta-cyclodextrin, allowed to dissolve and the complete feedstock is then added to 1×1 cm blister wells in 1 ml aliquots. These blisters are frozen at −70° C. and then loaded into a Heto freeze-drier (shelf held at −100° C.) and lyophilised for 4 h (ramping from −10° C. to 30° C.) to produce non-friable flexible porous wafers. The other half of the primary feedstock is cast onto a heated metal plate (40° C.) and rapidly microwave-dried to produce a thin flexible film which is then cut into individual dosages of 1×1 cm.

### Example 3

The following six different ion paired insulin/pullulan formulations listed in Table 1 below were assessed in a diabetic pig model:

| Reference | Description | Composition |
|---|---|---|
| 197 | DLPG as ion-pairer; | DLPG (3.3%); Brij-35 (10%); |
| | pH 3; Insulin Dose = | glycine (5%); pullulan(51.7%); |
| | 5 mg | trehalose(25%); insulin(5%) |
| 198 | SDS as ion-pairer; | SDS (1.5%); Brij-35 (10%); |
| | pH 3; Insulin Dose = | glycine (5%); Pullulan (53.5%); |
| | 5 mg | trehalose (25%); insulin (5%) |
| 201 | SDS as ion-pairer; | SDS (2.98%); Brij-35 (18%); |
| | pH 3; Insulin Dose = | glycine (5%); pullulan(49.0%); |
| | 10 mg | trehalose (15%); insulin (10%) |
| 202 | non ion-paired | SDS(2.5%); Brij-35(1.8%); |
| | formulation; pH 7.3; | Na glycocholate (2.5%); |
| | Insulin Dose = 5 mg | Pullulan (62.2%); |
| | | trehalose(25%); insulin (5%); |
| | | disodium hydrogen phosphate (1.0%) |
| 218 | TCA as ion-pairer; | TCA(4.63%); Brij-35 (10%); |
| | pH 3; Insulin Dose = | glycine (5%); Pullulan (50.4%); |
| | 5 mg | trehalose (25%); insulin (5%) |
| 256 | pH buffer is tartaric | SDS (1.5%); Brij-35 (10%); |
| | acid; | tartaric acid (10%); |
| | pH 3; Insulin Dose = | Pullulan (48.5%); |
| | 5 mg | trehalose (25%); insulin (5%) |
| Placebo | Placebo (no insulin) | Brij-35 (10%); glycine (5%); |
| | pH 3 | pullulan (60.0%); trehalose (25%) |
| SC | Insulin solution; | Solution: 3 mg/ml insulin in PBS. |
| Control | pH 7.3 | Inject 333 μl for 1 mg of insulin. |
| | Insulin Dose = 1 mg | |
| | Sub-cut | |
| | administration | |

Brij 35 = Polyoxyethylene(23)lauryl ether
DLPG = -2-dilauroyl-sn-glycero-3-phosphochoglycerol, sodium salt
SDS = Lauryl sulphate, sodium salt
TCA = Taurocholic acid

The placebo formulation consisted of the ion pairing primary surfactant, the secondary surfactant, pullulan and trehalose.

The SC Control was formulated in a phosphate buffered saline solution. Each pig was injected with 1 mg of insulin, which was equivalent to the amount of insulin being administered to the pig if two tablets of formulations 197, 198, 218, 256, 202 and one tablet of formulation 201 were administered and a 10% bioavailability was achieved.

Three separate solutions were prepared to be assessed.
Solution A—the insulin solution, also containing glycine, HCl, pullulan and trehalose,
Solution B—the secondary surfactant (Brij35)
Solution C—the ion pair agent in solution/dispersion (DLPG was warmed to 35 degrees Celsius, with magnetic stirring in a heating block).

Solution A was homogenised while solution C was added dropwise via a peristaltic pump. Rate of addition was approximately 2 ml a minute. At this point, the pH is adjusted to 3, by the dropwise addition of 5M HCl.

Finally, Solution A+C was gently stirred by the homogeniser, while Solution B is added dropwise by pastette.

The final A+C+B solution was dispensed into labelled blister trays using a displacement pipette. Blister trays were then placed in a pre-cooled freeze-dryer and then subjected to freeze-drying.

A diabetic* pig model was used to assess the concept that the formulations would increase the insulin concentration in diabetic pigs.
Pigs: Eight healthy (non-diabetic) domestic male pigs of approximately 40 kg were equipped with permanent sampling catheters in the jugular vein and the carotid artery. The pigs were treated with Streptozotocin to render them diabetic. An eight by eight (eight pigs by eight administrations) experimental design was utilised.
Number of formulations to assessed: Six insulin tablet formulations were assessed. One placebo tablet formulation was assessed as a negative control. One subcutaneous injection of insulin was assessed as a positive control.
Tablet Administration: Two tablets were administered on each administration day by diluting the tablet with 0.8 to 1.0 ml of water. This viscous fluid was draw up into a 1 ml syringe. The fluid was squirted in the buccal mucosa area of each pig mouth.
Testing: The test procedure was conducted on Mondays, Wednesdays, and Fridays, with the pigs in the overnight fasting condition.
Sampling: Two baseline blood samples at −30 and −10 minutes were taken. Tablet administration took place at 0 minutes. Blood sampling for the formulations took place at +5, +10, +15, +30, +60, +90, +120, +240, +360 minutes. For the subcutaneous control an additional blood samples at +480 minutes was taken. The blood samples were analysed for glucose concentration using. Plasma samples will be analysed for insulin concentration.

The pigs are rendered diabetic by the administration of Streptozotocin, which results in a Type I/Type II hybrid (i.e. the pigs are left with a small natural level of endogenous insulin which negates the need to administer any additional insulin to keep them alive).

1. Results from Pig Model

Table 2 contains the mean and standard deviation for the blood glucose levels versus time for the formulations assessed in the diabetic pig model.

TABLE 2
Mean and Standard Deviation of Blood Glucose
Levels from the Diabetic Pig Model

| | Mean Glucose Level (mmol/L) for Formulations | | | | | | | |
|---|---|---|---|---|---|---|---|---|
| | | | | | | | | SC |
| Time | 197 | 198 | 201 | 202 | 218 | 256 | Placebo | Control |
| −30 | 23.1 | 22.7 | 23.1 | 22.5 | 21.5 | 23.3 | 23.5 | 22.9 |
| | (0.8) | (2.8) | (4.5) | (3.5) | (4.5) | (3.5) | (2.4) | (2.5) |
| −10 | 22.3 | 21.1 | 22.4 | 20.8 | 20.2 | 22.7 | 22.6 | 21.9 |
| | (1.5) | (2.6) | (4.3) | (3.8) | (3.2) | (3.2) | (3.0) | (2.4) |
| 5 | 22.1 | 19.8 | 21.8 | 21.2 | 20.5 | 21.3 | 21.8 | 22.8 |
| | (2.8) | (2.5) | (4.0) | (4.1) | (3.3) | (3.6) | (2.1) | (3.6) |
| 10 | 22.8 | 19.3 | 22.1 | 21.2 | 20.7 | 21.6 | 21.2 | 21.1 |
| | (2.0) | (1.8) | (3.7) | (3.6) | (3.2) | (3.1) | (1.7) | (3.5) |
| 15 | 21.9 | 19.3 | 21.6 | 20.6 | 20.5 | 21.7 | 22.5 | 20.7 |
| | (2.5) | (3.3) | (3.7) | (3.2) | (3.4) | (2.3) | (2.5) | (5.2) |
| 30 | 22.1 | 20.4 | 21.5 | 20.6 | 20.6 | 21.8 | 22.0 | 19.1 |
| | (1.8) | (2.3) | (3.4) | (3.5) | (3.0) | (2.8) | (2.9) | (6.5) |
| 60 | 21.1 | 19.4 | 20.3 | 20.5 | 19.8 | 21.0 | 21.2 | 14.3 |
| | (3.2) | (2.0) | (3.3) | (4.2) | (3.5) | (2.9) | (3.1) | (7.9) |
| 90 | 19.7 | 18.8 | 19.9 | 19.9 | 19.3 | 20.6 | 20.6 | 11.9 |
| | (2.3) | (2.3) | (3.3) | (3.6) | (2.4) | (2.3) | (3.0) | (6.2) |
| 120 | 19.9 | 17.5 | 19.0 | 19.9 | 18.7 | 19.8 | 19.6 | 8.3 |
| | (1.7) | (2.9) | (2.8) | (4.1) | (2.2) | (2.1) | (2.2) | (6.6) |
| 240 | 18.9 | 18.8 | 16.7 | 18.6 | 17.8 | 18.2 | 19.0 | 7.1 |
| | (2.3) | (4.2) | (2.3) | (4.1) | (2.7) | (2.5) | (2.6) | (4.7) |
| 360 | 18.7 | 16.3 | 15.3 | 16.0 | 15.4 | 16.5 | 17.0 | 8.1 |
| | (3.8) | (2.8) | (2.4) | (3.2) | (3.1) | (2.0) | (1.7) | (4.0) |
| 480 | n/a | n/a | n/a | n/a | n/a | n/a | n/a | 10.9 |
| | | | | | | | | (4.3) |

( ) = standard deviation
n/a = blood glucose level not measured

Review of the above data highlights that the formulation 198 is showing a greater reduction in blood glucose level over the first 15 minutes post administration than with the subcutaneous injection. The data also highlights that formulations 198, 201 and 256 show a small additional reduction in blood glucose level when compared to placebo (1 to 9% less blood glucose at 60 minutes when compared to placebo, which should be related to the subcutaneous injection which has 32% less blood glucose than placebo).

The data also demonstrate that the placebo formulation has shown a reduction in blood glucose over time. Whilst the exact mechanism for this is unknown a probable cause is that the administration of the tablet has caused the formation of gastric juices in the pigs stomach which has caused a reduction in blood glucose levels over time. Due to this finding it was deemed appropriate to correct for this phenomenon by subtracting the placebo blood glucose level at each time point from the blood glucose level for each tablet formulation.

Review of this corrected data confirms the findings highlighted above (i.e. formulation 198 is showing a greater reduction in blood glucose level over the first 15 minutes post administration than with the subcutaneous injection and that formulations 198, 201 and 256 show a small additional reduction in blood glucose level when compared to placebo).

A small additional reduction in the blood glucose levels occurred with formulations 198, 201 and 256. The data shows for formulation 198 the maximum reduction in blood glucose level occurred 15 minutes post tablet administration, with a slight increase and levelling off of blood glucose levels 30 minutes post tablet administration. An increase in blood glucose level from 120 minutes onwards was observed. Formulations 201 and 256 show a different trend with the maximum reduction in blood glucose occurring at 240 minutes. The difference in glucose level trend between formulations 198 and 202 is most probably due to formulation 201 containing 10% insulin, whereas 198 contained 5% insulin, and hence an insulin “reservoir” effect may have occurred which caused the blood glucose levels to drop over an increased length of time.

### Example 4

### Alendronic Acid

Bisphosphonates are potent compounds used in the treatment of osteoporosis, Paget's disease and in the treatment of bone metastases and hypercalcaemia of malignancies. The most commonly used bisphosphonates are alendronic acid, clodronic acid and etidronic acid. Oral administration is associated with a number of severe side effects including oesophagitis, dyspepsia, diarrhoea, abdominal pain, oesophageal erosions and ulcerations. Further, oral bioavailability is in the range of 0.4 to 0.7% for alendronic acid and from to 1 to 6% for clodronic and etidronic acids. When administrated with food, bioavailability can be significantly reduced even to the level of being negligible. For alendronic acid, the usual daily dosage is 5 to 10 mg for osteoporosis and the dosage for Paget's disease is about 40 mg per day. The present invention provides an oromucosal dosage from which avoids the potentially serious side effects and the poor and erratic bioavailability observed from oral delivery.

Thus, alendronic acid is ion-paired with a suitable anionic surfactant, such as SDS or AOT at a suitably acidic pH of approximately 3 to 6 and is formulated into films or wafers as described in Examples 1 or 2 above.

### Example 5

The following are examples of HIP complexes that may be used in the invention and how they may be formulated.

| | | | | | | | Protein/Surfactant Mixing | | | | | |
|---|---|---|---|---|---|---|---|---|---|---|---|---|
| | Net Charge | | | | Ratio - Protein/Surfactant | | Protein | | | Surfactant | | |
| Protein | at low pH | M. Mass | Surfactant | M. Mass | Molar Ratio | Mass Ratio | mg | mg/ml | ml | mg | mg/ml | ml |
| Insulin | +6 | 5808 | AOT* | 445 | 4.5 | 2.90 | 100.0 | 2.00 | 50.0 | 34.5 | 10.0 | 3.45 |
| | | 5808 | SDS | 288 | 4.5 | 4.48 | 100.0 | 2.00 | 50.0 | 22.3 | 10.0 | 2.23 |
| | | 5808 | SDS | 288 | 6.0 | 3.36 | 100.0 | 2.00 | 50.0 | 29.8 | 10.0 | 2.98 |
| | | 5808 | DPPG | 745 | 8.0 | 0.97 | 100.0 | 2.00 | 50.0 | 102.6 | 10.0 | 10.26 |
| | | 5808 | DPPG | 745 | 4.0 | 1.95 | 100.0 | 2.00 | 50.0 | 51.3 | 10.0 | 5.13 |
| HGH(1-217) | +25 | 24849 | DPPG | 745 | 25 | 1.33 | 100.0 | 2.00 | 50.0 | 75.0 | 10.0 | 7.50 |
| | | 24849 | DPPG | 745 | 33 | 1.01 | 100.0 | 2.00 | 50.0 | 98.9 | 10.0 | 9.89 |
| Leuprolide | +2 | 1269.4 | DPPG | 745 | 2 | 0.85 | 100.0 | 2.00 | 50.0 | 117.4 | 10.0 | 11.74 |
| | | 1269.4 | DPPG | 745 | 2.5 | 0.68 | 100.0 | 2.00 | 50.0 | 146.7 | 10.0 | 14.67 |

1. A composition for oromucosal delivery of a biologically active agent, wherein said composition releases said biologically active agent within 5 minutes when applied to an oromucosal surface in use, wherein said composition comprises said biologically active agent and a matrix-former, and said biologically active agent is present as an ion pair complex.

2. A composition for oromucosal delivery of a biologically active agent, wherein said composition releases said biologically active agent within 5 minutes when applied to an oromucosal surface in use, wherein said composition comprises said biologically active agent and a matrix-former, and said composition is formed by freeze drying a solution comprising said biologically active agent and said matrix-former.

3. A composition according to claims 1 or 2, wherein said composition dissolves or disperses to release said agent.

4. A composition according to claims 1 or 2, wherein said biologically active agent is a protein.

5. A composition according to claim 4, wherein said protein is insulin.

6. A composition according to claims 1 or 2, wherein said matrix-former is pullulan.

7. A composition according to claims 1 or 2, which comprises a phospholipid.

8. A composition according to claims 1 or 2, which is in the form of a film or wafer.

9. A composition according to claims 1 or 2, which is mucoadhesive.

10. A composition according to claims 1 or 2, wherein said biologically active agent can be converted into an ionic form.

11. A method of producing a composition according to claims 1 or 2, comprising the steps of
a) forming a solution, dispersion or emulsion comprising said matrix-former and said biologically active agent; and
b) removing the a solvent.

12. A composition according to claims 1 or 2 for use in medicine.

13. A method for treating a condition or disorder which comprises administering an effective amount of a composition according to claims 1 or 2 to a patient in need thereof.

14. A method of using a composition according to claims 1 or 2 in the manufacture of a medicament for the treatment of a condition or disorder.

15. A method according to claim 13, wherein said condition or disorder is diabetes.

16. A method according to claim 14, wherein said condition or disorder is diabetes.
